(19) United States
(12) Patent Application Publication
(10) Pub. No.: US 2002/0091397 A1
(43) Pub. Date: Jul. 11, 2002

(54) Structure of intestinal anastomosis gun

(76) Inventor: Te-Chuan Chen, Taipei (TW)

Correspondence Address: BACON & THOMAS, PLLC, 4th Floor, 625 Slaters Lane, Alexandria, VA, 22314-1176, US

(21) Appl. No.: 09/754,200

(22) Filed: Jan. 5, 2001

(51) Int. Cl. A61B 17/08

(52) U.S. Cl. 606/153

(57) ABSTRACT

An intestinal anastomosis gun is disclosed including a gun barrel, a gun head unit formed of a push rod and a hollow anastomosis rod, and an operating mechanism formed of a rotary member, a center shaft and a hollow screw cap. The center shaft and the anastomosis rod are moved axially upon rotary motion of the rotary member, thereby causing the head of the anastomosis rod to open or close. The intestinal anastomosis gun is inserted through the anus into the intestinal canal of the large intestine or rectum after installation of an anastomosis ring and a synthetic detour tube, enabling the synthetic detour tube to be fastened to the intestinal canal during an intestinal detour operation.

### BACKGROUND OF THE INVENTION

1. Field of the Invention

The present invention relates to intestinal operation instrument and, more particularly, to an improved structure of intestinal anastomisis gun for operating an anastomosis ring.

2. Description of Related Art

Biofragentable anastomosis ring, which is also called BAR or Valtrac, is a product of Davis & Geck, American Cyanamid Company Danbury, Conn. 06810. This biofragentable anastomosis ring has become more and more popularly accepted because it is safe in use in an intestinal anastomosis and decomposable within about 2˜3 weeks for discharging out of the body with stool.

U.S. Pat. No. 5,464,415 discloses a sutureless intestinal anastomosis gun convenient for use in intestinal anastomosis.

### SUMMARY OF THE INVENTION

The intestinal anastomosis gun of the present invention comprises a gun barrel, a gun head unit, and an operating mechanism. The gun barrel comprises a first endpiece, and a second endpiece. The gun head unit is comprised of a hollow anastomosis rod, and a push rod. The hollow anastomosis rod comprises a head and a tail. The push rod is fastened to the first endpiece of the gun barrel and inserted into the anastomosis rod. The operating mechanism comprises a rotary member, center shaft, and a hollow screw cap. The hollow screw cap is fastened to the second endpiece of the gun barrel. The intestinal anastomosis gun eliminates the use of a quick connector. The center shaft is directly fastened to the hollow anastomosis rod by a screw joint. The gun barrel is made in integrity, and fits any of a variety of anastomosis rings of different sizes. Because the center shaft is directly fastened to the hollow anastomosis rod by a screw joint, the anastomosis action is direct and accurate.

The diameter of the first endpiece of the gun barrel is smaller than regular anastomosis rings and the gun barrel of the prior art intestinal anastomosis gun, the intestinal anastomosis gun can easily smoothly be inserted into the intestinal canal. The intestinal anastomosis gun of the invention is light, convenient, accurate and reliable in use. When rotating the rotary member, the center shaft and the anastomosis rod are moved axially downwards or upwards, causing the head of the anastomosis gun to open or to close.

If the large intestine or the rectum suffers a cancer or disease and must receive an operation, the part in trouble of the large intestine or rectum is cut off, and then the intestinal anastomosis gun of the present invention is used. One end of the flexible, thin layer of synthetic detour tube is tied to the neck of the anastomosis ring, and then the other end of the detour tube is sleeved onto the gun barrel over the gun head unit, and then the intestinal anastomosis gun is inserted through the bottom opening of the anus or large intestine into the intestinal canal, and then the anastomosis ring is operated to secure the synthetic detour tube to the normal large intestine, for enabling excrement to be discharged out of the anus through the synthetic detour tube before the operation area is well cured. Because the synthetic detour tube and the anastomosis ring are well-known medical accessories and not within the scope of the claims of the present invention, they are not described hereinafter in detail. The use of the present invention is simple, safe, and accurate. It eliminates the operation of making a temporary opening on the large intestine and the operation of closing of the temporary opening.

### BRIEF DESCRIPTION OF THE DRAWINGS

FIG. 1 is a perspective, partially cutaway view of an intestinal anastomosis gun according to the present invention.

FIG. 2 is an exploded view of the intestinal anastomosis gun according to the present invention.

FIG. 3 is a sectional assembly view of the intestinal anastomosis gun according to the present invention.

FIG. 4 illustrates the action of the intestinal anastomosis gun according to the present invention.

FIG. 5 illustrates a synthetic detour tube fastened to the intestinal anastomosis gun according to the present invention.

FIG. 6 illustrates the intestinal anastomosis gun aimed at the intestinal canal according to the present invention.

FIG. 7 illustrates the anastomosis ring and the synthetic detour tube fastened to the large intestine according to the present invention.

FIG. 8 illustrates the rotary member rotated, the head of the anastomosis rod stretched open according to the present invention.

FIG. 9 illustrates the head of the anastomosis rod opened and pressed on the anastomosis ring according to the present invention.

FIG. 10 illustrates the rotary member rotated in the reversed direction, the head of the anastomosis rod released from the pressure according to the present invention.

FIG. 11 illustrates the intestinal operation completed, the intestinal anastomosis gun removed from the anastomosis ring and the synthetic detour tube according to the present invention.

### DETAILED DESCRIPTION OF THE PREFERRED EMBODIMENT

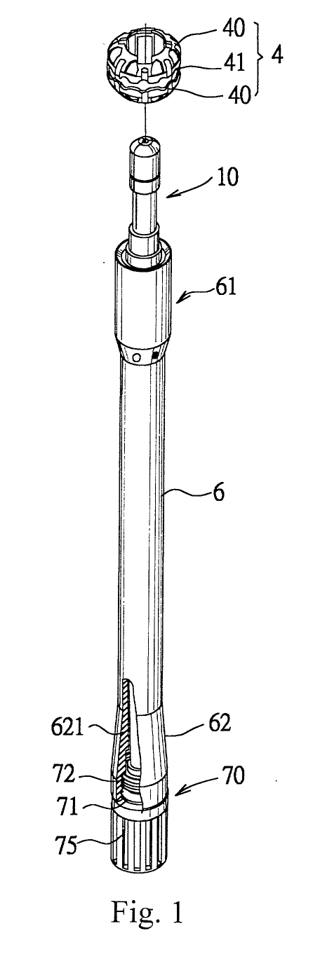

With reference to FIG. 1, there is shown an intestinal anastomosis gun comprised of a gun barrel 6, a gun head unit 10, and an operating mechanism 70.

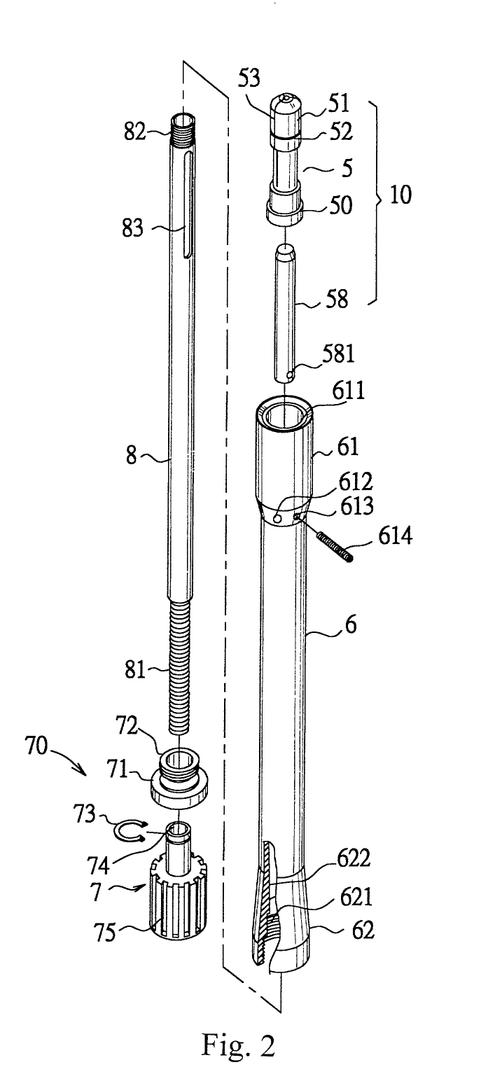

Referring to FIG. 2, the gun barrel 6 has a first endpiece 61 and a second endpiece 62. The first endpiece 61 of the gun barrel 6 comprises an annular groove 611 curved inwards at the top side thereof and adapted to hold an anastomosis ring 4, and a plurality of round holes 612 radially disposed at the bottom side thereof in communication with the inside space of the gun barrel 6 and adapted to drain off gas and fluid after insertion of the gun barrel 6 into the large intestine. The second endpiece 62 of the gun barrel 5 comprises an internal thread 621 and a receiving chamber 622.

Referring to FIG. 2 again, the gun head unit 10 comprises a push rod 58, and a hollow anastomosis rod 5. The anastomosis rod 5 has a head 51 and a tail 50. The tail 50 is internally threaded. The anastomosis rod 5 is made of flexible material. Longitudinal splits 53 are provided at the anastomosis rod 5 to enhance the spring power of the anastomosis rod 5 in radial direction. A binding ring 52 is mounted on the head 51 of the anastomosis rod 5 to enhance the spring power of contraction of the head 51. The push rod 58 has one end, namely, the top end inserted into the inside of the anastomosis rod 5, and an opposite end, namely, the bottom end inserted into the hollow top end of the center shaft 8 of the operating mechanism 70. A screw 614 is threaded into a radial screw hole 613 on the bottom side of the first endpiece 61 of the gun barrel 6 and a radial screw hole 581 on the bottom end of the push rod 58 and inserted into a longitudinal sliding slot 83 to secure the center shaft 8 to the inside of the gun barrel 6 and to limit axial movement of the center shaft 8 relative to the gun barrel 6 within a distance equal to the length of the longitudinal sliding slot 83. The structure and application of the anastomosis ring 4 are apparent to those skilled in the art. The anastomosis ring 4 shown in the annexed drawings is formed of two symmetrical halves 40 and 40′. The anastomosis ring 4 has a neck 41.

Referring to FIG. 2 again, the operating mechanism 70 comprises a rotary member 7, a center shaft 8, and a hollow screw cap 71. The screw cap 71 is threaded into the internal thread 621 of the second endpiece 62 of the gun barrel 6. The rotary member 7 is a stepped cylindrical member coupled to the screw cap 71, comprising a knob 75 disposed at the rear end thereof and stopped at one side of the screw cap 71 outside the second endpiece 62 of the gun barrel 6, and an axial center screw hole 74 extended through two distal ends thereof.

Referring to FIG. 2 again, the center shaft 8 comprises an outer thread 82 disposed at the top end thereof and threaded into the internally threaded tail 50 of the anastomosis rod 5, a screw rod 81 axially extended from the rear end thereof and threaded into the axial center screw hole 74 of the rotary member 7. Rotating the rotary member 7 causes the center shaft 8 to be moved axially in the gun barrel 6. A C-shaped retainer ring 73 is fastened to the top end of the rotary member 7 to secure the rotary member 7 to the screw cap 71. The screw cap 71 has a threaded shank 72 threaded into the internal thread 621 of the second endpiece 62 of the gun barrel 6. The knob 75 of the rotary member 7 is peripherally longitudinally toothed for gripping of the hand positively.

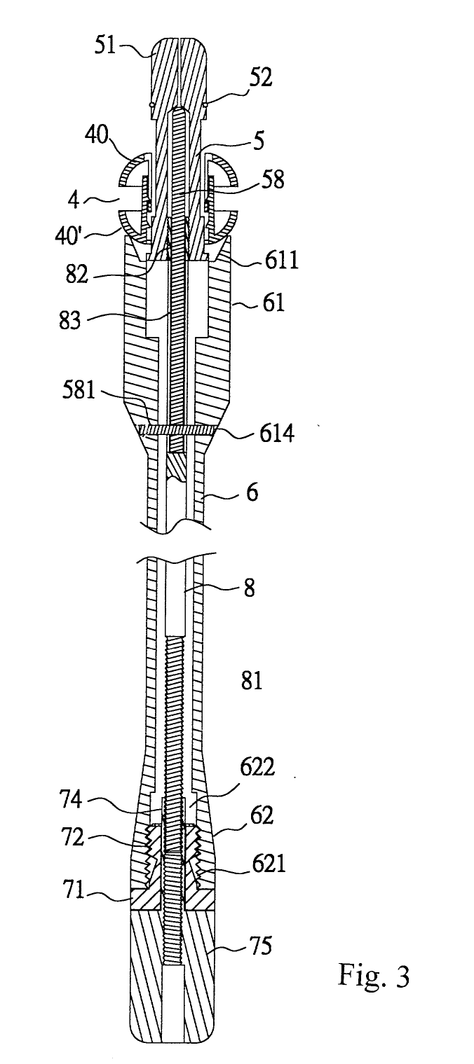

Referring to FIG. 3, the threaded shank 72 of the screw cap 71 is threaded into the internal thread621 of the second endpiece 62 of the gun barrel 6, and the outer thread 82 of the center shaft 8 is threaded into the internally threaded tail 50 of the anastomosis rod 5.

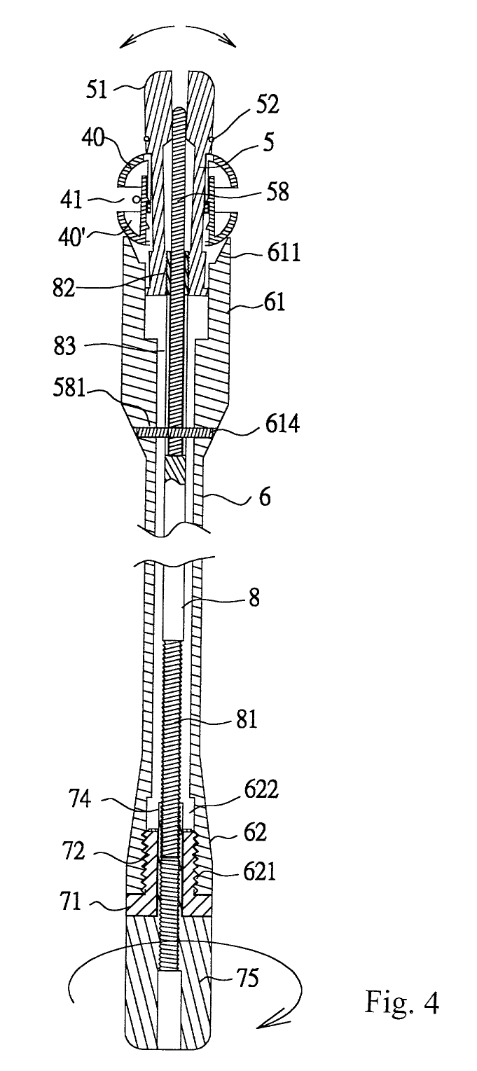

Referring to FIG. 4 and FIG. 3 again, rotating the rotary member 7 causes the center shaft 8 to move the anastomosis rod 5 axially upwards or downwards. As illustrated in FIG. 4, when the anastomosis rod 5 is lowered, the top end of the anastomosis rod 5 is stretched radially outwards by the push rod 58 and secured to the anastomosis ring 4. As illustrated in FIG. 3, when the anastomosis rod 5 is lifted, the top end of the anastomosis rod 5 is released from the pressure of the push rod 58 and, immediately returns to its former shape by means of the effect of its material spring power.

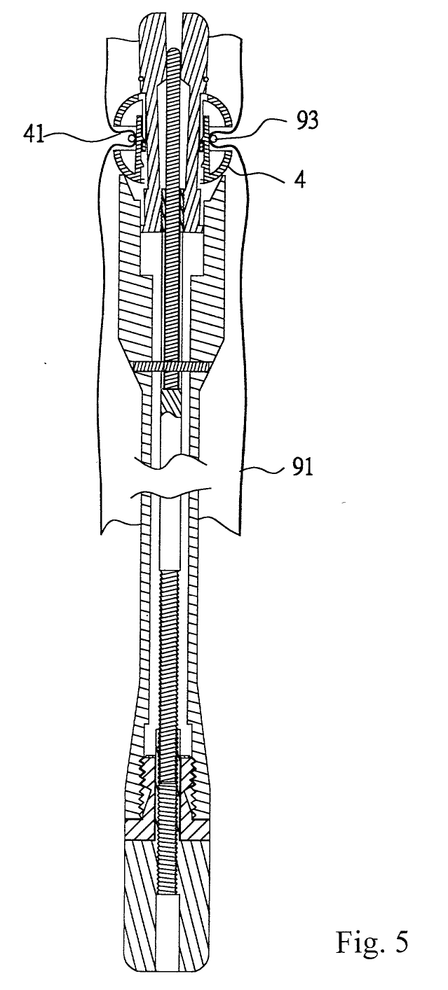

Referring to FIG. 4 again, during the surgical operation, the rotary member 7 is rotated to move the center shaft 8 and the anastomosis rod 5 downwards, to further cause the top end of the anastomosis rod 5 to be stretched radially outwards by the push rod 58. When stretched radially outwards, the head 51 of the anastomosis rod 5 holds down the symmetrical halves 40 and 40′ of the anastomosis ring 4 slightly. As illustrated in FIG. 5, the synthetic detour tube 91 is sleeved onto the first endpiece 61 of the gun barrel 6, and the upper end of the synthetic detour tube 91 is fixedly fastened to the neck 41 of the anastomosis ring 4 by a tie string 93.

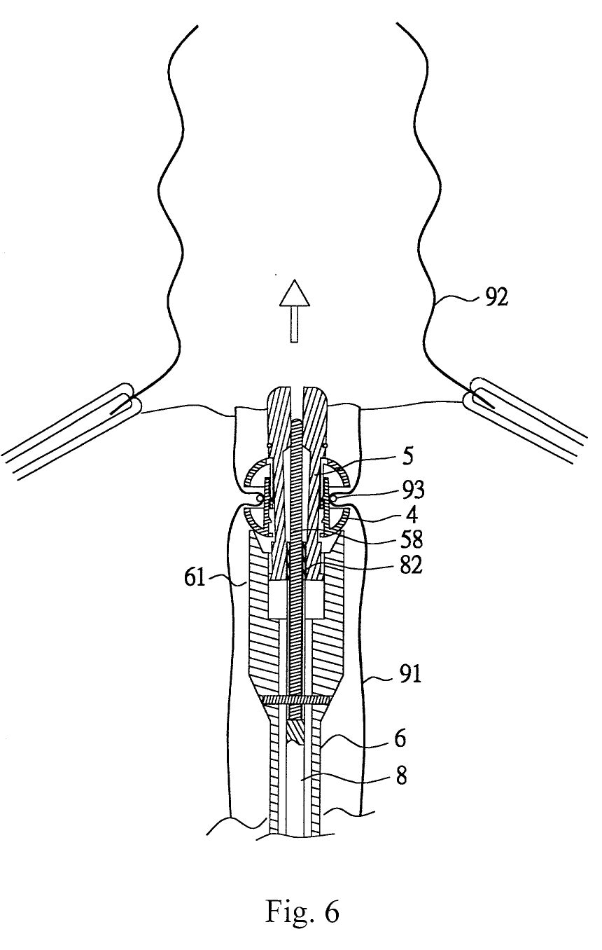

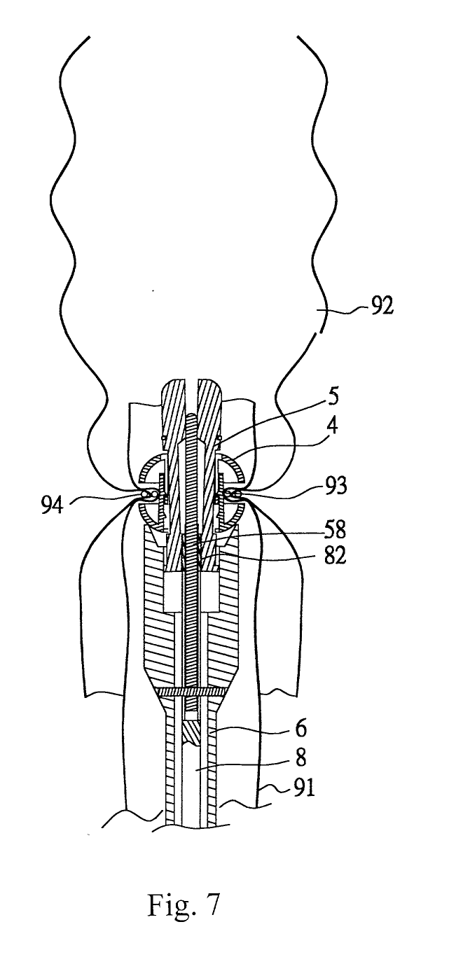

Referring to FIGS. 6 and 7, the intestinal anastomosis gun with the synthetic detour tube 91 and the anastomosis ring 4 are inserted through the opening of the intestinal canal 92 into a certain distance inside the intestinal canal 92, and then a tie string 94 is wound round the outside wall of the intestinal canal 92 and fastened to the neck 41 of the anastomosis ring 4.

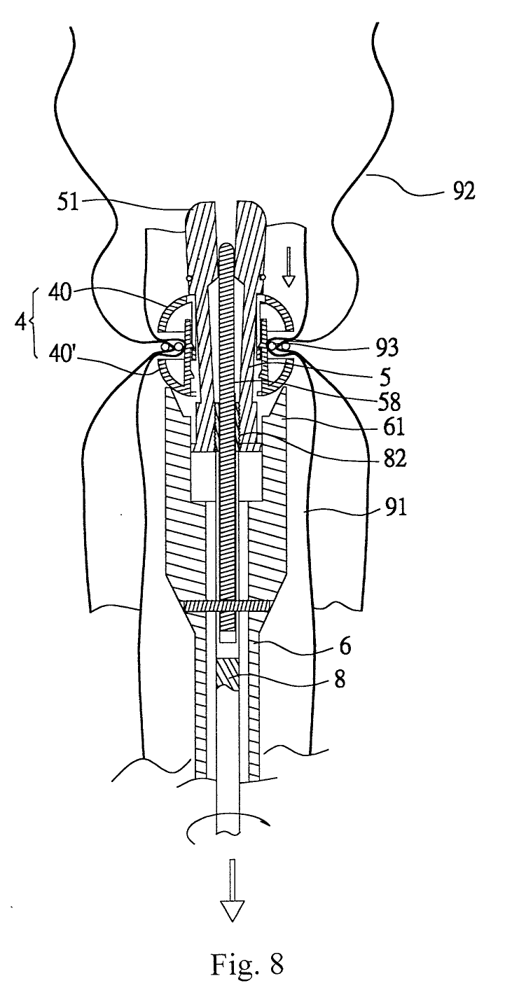

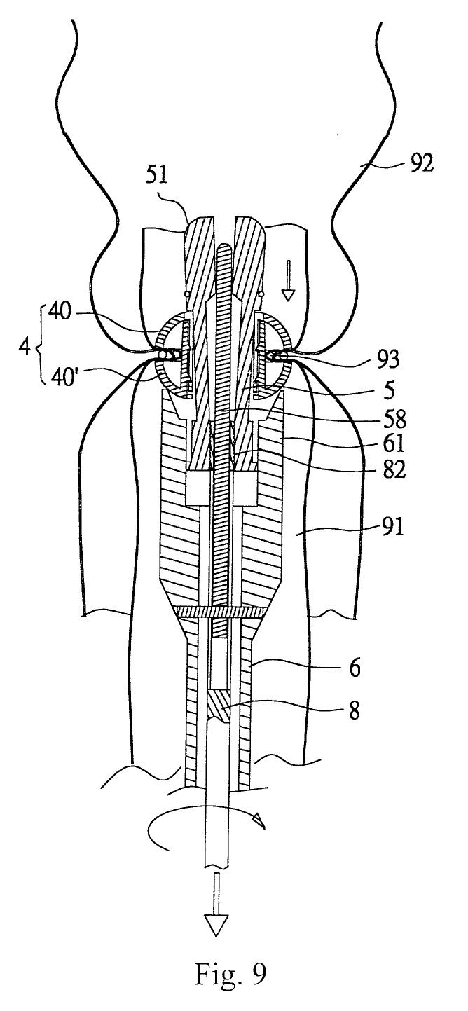

Referring to FIGS. 8 and 9, the rotary member 7 is rotated to lower the center shaft 8 and the anastomosis rod 5, causing the head 51 of the anastomosis rod 5 to be stretched open by the push rod 58 to further hold down the symmetrical halves 40 and 40′ of the anastomosis ring 4, and therefore the intestinal canal 92, the synthetic detour tube 91 and the anastomosis ring 4 are positively fixed in position.

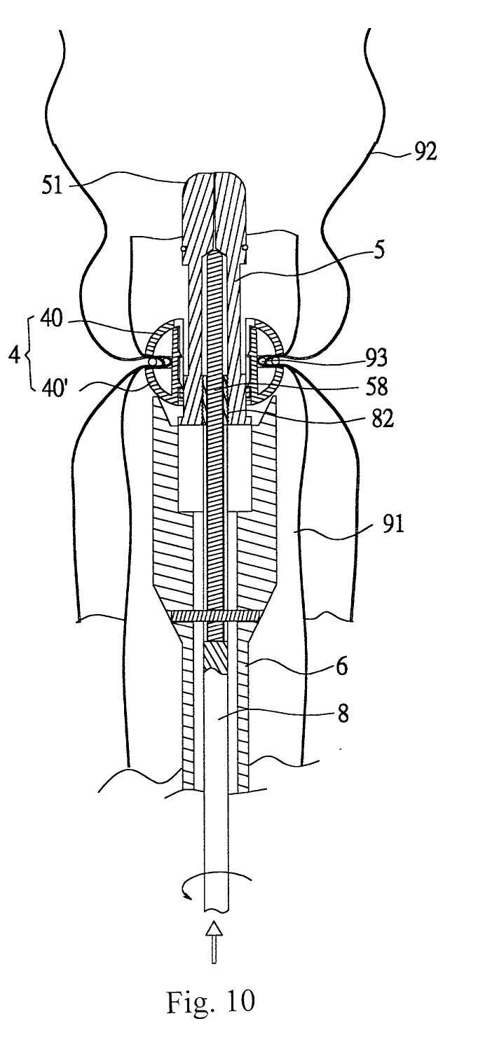

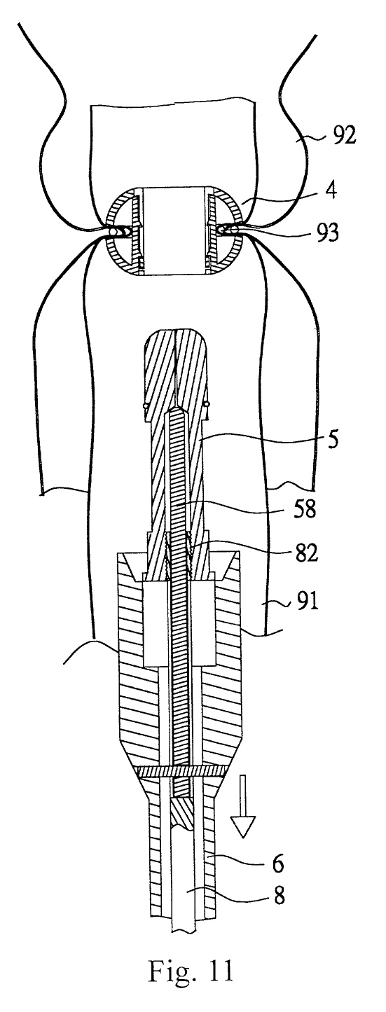

Referring to FIGS. 10 and 11, the rotary member 7 is rotated in the reversed direction to lift the center shaft 8 and the anastomosis ring 5 after fixation of the intestinal canal 92, the synthetic detour tube 91 and the anastomosis ring 4, so as to release the anastomosis rod 5 from the anastomosis ring 4, enabling the intestinal anastomosis gun to be removed from the body of the patient. Thus, the operation of the intestinal canal 92 is done.

Although the present invention has been explained in relation to its preferred embodiment, it is to be understood that many other possible modifications and variations can be made without departing from the spirit and scope of the invention as hereinafter claimed.

What is claimed is:

1. An intestinal anastomosis gun comprising:
a gun barrel, said gun barrel comprising a first endpiece and a second endpiece;
a gun head unit, said gun head unit comprised of a hollow anastomosis rod, said hollow anastomosis rod comprising a head and a tail, and a push rod fastened to the first endpiece of said gun barrel and inserted into said anastomosis rod; and
an operating mechanism, said operating mechanism comprising a center shaft inserted into said gun barrel, said center shaft having a top end connected to the tail of said hollow anastomosis rod, a hollow screw cap fastened to the second endpiece of said gun barrel, and a rotary member;
wherein rotating said rotary member causes said center shaft and said push rod to be moved axially downwards/upwards in said gun barrel relative to said hollow anastomosis rod to stretch open/release the head of said hollow anastomosis rod.

2. The intestinal anastomosis gun as claimed in claim 1, wherein said hollow anastomosis rod is made of flexible material having a plurality of longitudinal splits and a binding ring fastened to the head thereof.

3. The intestinal anastomosis gun as claimed in claim 1, wherein said center shaft has a bottom end terminating into a screw rod and threaded into a screw hole on said rotary member such that rotating said rotary member causes said center shaft to be moved axially.

4. The intestinal anastomosis gun as claimed in claim 1, wherein said the first endpiece of said gun barrel comprises an annular groove adapted to hold an anastomosis ring.

5. The intestinal anastomosis gun as claimed in claim 4, wherein said anastomosis ring is formed of two symmetrical halves, having a neck.
